(19) United States
(12) Patent Application Publication
(10) Pub. No.: US 2021/0285085 A1
(43) Pub. Date: Sep. 16, 2021

(54) Optimum Surface Texture Geometry

(71) Applicant: PULSE IP, LLC, Quakertown, PA (US)

(72) Inventor: Andrew E. Fisk, Philadelphia, PA (US)

(21) Appl. No.: 16/880,481

(22) Filed: May 21, 2020

Related U.S. Application Data
(63) Continuation of application No. 13/087,435, filed on Apr. 15, 2011, now abandoned.
Application No. 13/087,435 is a division of application No. 11/868,808, filed on Oct. 8, 2007, now abandoned.
Application No. 11/868,808 is a continuation-in-part of application No. 11/754,601, filed on May 29, 2007, now abandoned.

(51) Int. Cl. C23C 14/00; C23C 14/06; C23C 14/54

(52) U.S. Cl. CPC C23C 14/0036; A61N 1/05; C23C 14/541; C23C 14/0641

(57) ABSTRACT

A surface geometry for an implantable medical electrode that optimizes the electrical characteristics of the electrode and enables an efficient transfer of signals from the electrode to surrounding bodily tissue. The coating is optimized to increase the double layer capacitance and to lower the after-potential polarization for signals having a pulse width in a pre-determined range by keeping the amplitude of the surface geometry with a desired range.

### CROSS REFERENCE TO RELATED APPLICATIONS

This application is a divisional of co-pending U.S. application Ser. No. 11/868,808, filed Oct. 8, 2007, entitled “Optimum Surface Texture Geometry,” which is a continuation-in-part of co-pending U.S. application Ser. No. 11/754,601, filed May 29, 2007, entitled “Method for Producing A Coating With Improved Adhesion”

### FIELD OF THE INVENTION

This invention relates generally to an optimized surface geometry for electrically active medical devices, and, in particular, to the surface geometry for devices intended to be permanently implanted into the human body for use as stimulation electrodes.

### BACKGROUND OF THE INVENTION

Active implantable devices are typically electrodes used for the stimulation of tissue or the sensing of electrical bio-rhythms. Typically, the electrical performance of implantable electrodes can be enhanced by applying a coating to the external surfaces, to provide an electrically optimized interface with the tissues of the body with which the electrode is in contact. It is known that the application of a coating having a high surface area or a highly porous coating to an implantable electrode increases the double layer capacitance of the electrode and reduces the after-potential polarization, thereby increasing device battery life, or allowing for lower capture thresholds and improved sensing of certain electrical signals, such as R and P waves. A reduction in after-potential polarization results in an increase in charge transfer efficiency by allowing increased charge transfer at lower voltages. This is of particular interest in neurological stimulation. Double layer capacitance is typically measured by means of electrochemical impedance spectroscopy (EIS). In this method an electrode is submerged in a electrolytic bath and a small (10 mV) cyclic wave for is imposed on the electrode. The current and voltage response of the electrode/electrolyte system is measured to determine the double layer capacitance. The capacitance is the predominant factor in the impedance at low frequencies (<10 Hz) and thus the capacitance is typically measured at frequencies of 0.001 Hz-1 Hz.

Such coatings, in addition to a having a large surface area and being biocompatible and corrosion resistant in bodily fluids, must strongly adhere to the substrate (the electrode surface) and have good abrasion resistance, showing no signs of flaking during post-coating assembly and use. Adhesion of an electrode coating is of critical interest since the flaking of a coating during implant can cause infection and flaking of the coating post-implant can cause a sudden increase in the charge required to stimulate tissue. Additionally, it is undesirable to have a brittle surface or a surface prone to abrasion, as materials abraded from the surface may have negative effect on the electrical performance of the device and cause tissue scaring or inflammation.

Coatings having large surface areas are produced as porous deposits having morphologies described as columnar or cauliflower in structure. Such coatings may be deposited on the surface of the electrode by any means well known in the art, such as by physical vapor deposition or sputtering. It is known in various examples of prior art that an increase in porosity leads to an increase in the double layer capacitance. Prior art in the areas of super capacitors, electrolytic batteries and fuel cells have show great improvements by interconnected networks of porosity.

The parent application, Ser. No. 11/754,601, discloses a method for producing a coating having high surface area and exhibiting low after-potential polarization, while retaining good adhesion characteristics, and is incorporated herein in its entirety.

However, it has been found that, when used for the electrical stimulation of cellular tissue, such as in cardiac or neural stimulation, the increase in porosity and/or surface area, and therefore double layer capacitance measured by electrochemical impedance spectroscopy (EIS), does not necessarily produce the expected result of lowering the after-potential polarization of the electrode or increasing the charge transfer capability of the electrode.

Porous structures such as those found in the prior art applied to batteries, capacitors and fuel cells are subjected to long charge and discharge times on the order of several seconds in some cases. Therefore the rate of voltage change is in the order of 1 V/s-100 V/s. However, in the case of a medical electrode for stimulation and sensing of biorhythms, the pulse duration must be as short as possible to limit the voltage differential across the tissue and prevent hydrogen formation at the electrode surface. Voltage sweep rate changes for a medical electrode are on the order of 1×10\hat{ }2-1×10\hat{ }6 V/s.

By applying a common porosity transmission model to the electrode model it was observed that for the region of tissue stimulation, the diffusional properties of a porous structure do not allow the charging and discharge of the double layer capacitance formed within the porous structure. It is found in the present invention that the increase in micro-porosity has no effect on the electrical stimulation efficiency of an implantable medical electrode.

The problem is shown diagrammatically in FIG. 9. The double layer capacitance can be modeled by resistor/capacitor pairings along all surfaces of the coating layer. However, added resistance, represented by resistors Rₛ₁, Rₛ₂, Rₛ₃ and Rₛ₄ in the porous areas between the columns, is also present. For very short charge and discharge rates, the added resistance between the columns tends to dominate the resistor/capacitor pairs, preventing the charging and discharging of those RC pairings between the columns. This leaves only those resistor/capacitor pairings present at the tops of the columns (not shown) to transfer signals from the electrode to the cells of the body. As a result, the efficiency of the signal transfer is compromised.

The desirable characteristics of the coating, those being high double layer capacitance of the electrode and a low after-potential polarization effect, are enhanced when the surface area of the coating is increased. In order to maximize the electrical performance of a medical electrode the surface area of the electrode must be maximized without regard to the porosity.

### SUMMARY OF THE INVENTION

The present invention meets these objectives by disclosing an optimized surface geometry for an implantable medical electrode, which optimizes the electrical performance of the electrode while mitigating the undesirable effects associated with prior art porous surfaces.

It is known that the method for charge transfer in a medical electrode is by the charging and discharging of the electrical double layer capacitance formed on the surface of the electrode. This layer can be thought of as a simple parallel plate model in which the tissue to be stimulated is separated from the electrode surface by a barrier consisting primarily of water, Na, K and Cl. The thickness of this layer is dictated by the concentration of the electrolyte in the body and is therefore uniform over the working life of the electrode. The thickness of an electrical double layer formed by an electrical conductor in 0.9% saline (i.e., body fluid) is on the order of 1 nm and the expected thickness of the double layer capacitance formed in normal body electrolyte would be 0.5 nm-10 nm, more typically about 5-6 nm.

A typical human cell is on the order of 5,000 nm-10,000 nm in size. Because the cells are much larger than the layer and much smaller than the electrode surface it can be though of as being parallel to the surface of the electrode. As the non-polarized electrolyte (the electrolyte present but not participating in the electrical double layer) increases, the impedance of the tissue-electrode system increases. This is known as the solution resistance in electrochemical terms. The increased impedance results in a less effective charge transfer due to a dissipation of voltage along the solution resistance path. To minimize this impedance, the tissue to be stimulated should be as close to the electrode surface as possible. It would therefore be preferred, for these purposes, to have the electrode surface flat and parallel to the tissue.

Since the two optimum characteristics for low solution resistance and high double layer capacitance are in conflict, it is found that an optimum geometry consists of an angled, repeating surface texture. In a 2D representation this would be a saw tooth pattern with a amplitude equal to ½ wavelength. In a 3D representation the optimum geometry would be a surface having a repeating pyramidal geometry with all sides of the pyramids being of equal length. The base of the pyramidal shape is preferred to be trilateral to increase the number of structures present in any given area, but may be quadrilateral or other polygonal shape.

The optimal amplitude of the pyramidal-shaped surface structures is dictated by the rate of charge and discharge of the double layer capacitance, which in turn is dictated by the stimulation waveform. In the case of cardiac and neurological stimulation, this waveform is typically 0.5 ms-5 ms in duration, which suggests an optimal geometric amplitude of 70 nm-750 nm for the trilateral pyramidal pattern and 25 nm-350 nm for the quadrilateral pyramidal pattern.

In the preferred method, the surface geometry pattern was introduced onto the electrode by means of a coating. The coating used consisted of a TiN film deposited in such as way as to produce a columnar structure with a highly orientated [1,1,1] crystal texture. It is known that the NaCl type crystal structure of TiN results in a pyramidal surface morphology when deposited in singular columns with a [1,1,1] texture. This method is explained in full in the parent application.

Surface textures may also be formed by means other than PVD coating, such as by utilizing a laser to etch the surface details by removal of material, should produce the same results.

Experiments involving changes in deposition parameters resulting in changes in the width of the crystallite grains, which in turn varies the amplitude of the surface geometry, were performed to confirm. the expected optimum geometry. The factors effecting the width of the gains is well known and described in the prior art and is a adatom mobility.

### DESCRIPTION OF THE DRAWINGS

FIG. 1 shows a surface having crystallites of 70-100 nm amplitude.

FIG. 2 shows a surface according to the preferred embodiment of the invention, having crystallites of 200-400 nm amplitude.

FIG. 3 shows a surface having crystallites of 500-1200 nm amplitude.

FIG. 4 Shows the results of Trial 7 which resulted in crystallites of 150-350 nm amplitude.

FIG. 5 shows a surface having crystallites of 200 nm-300 nm amplitude and a >90% preferred crystal orientation of [1,1,1].

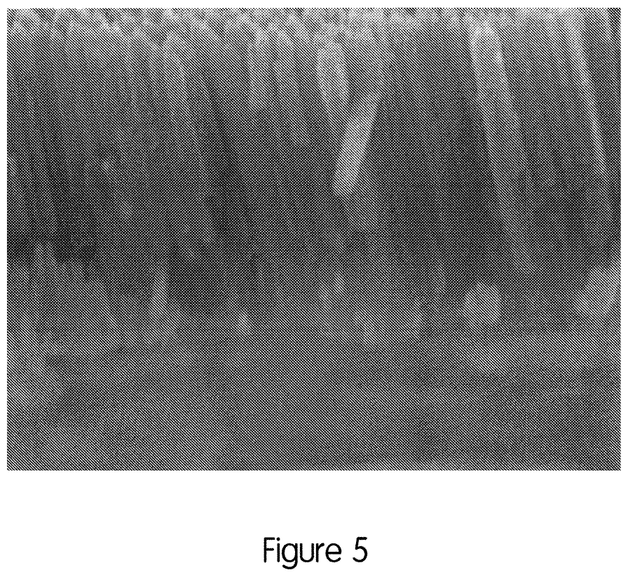

FIG. 6 shows a surface according to the preferred embodiment of the invention, having crystallites of 200 nm-350 nm amplitude and a >90% preferred crystal orientation of [1,1,1]

FIG. 7 is a graph showing both after-potential polarization and double layer capacitance as a function of the geometric amplitude of the crystallites for various stimulation pulse widths.

FIG. 8 is a plot of a stimulation pulse showing the effect of after-potential polarization.

FIG. 9 is a 2D representation of the saw tooth geometry of the surface with an electrical double layer made of Na and Cl ions. This figure is not to scale.

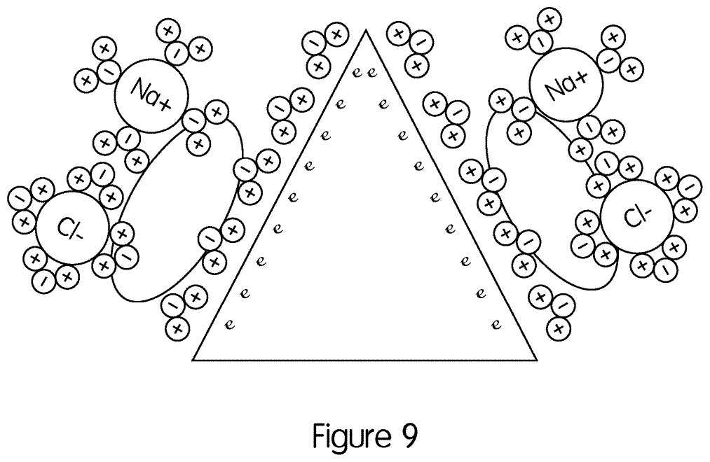

FIG. 10 shows a typical pore transmission line model showing increasing impedance (R) as a function of the porosity between columns.

### DETAILED DESCRIPTION OF THE INVENTION

The present invention realizes a performance advantage over typical prior art surface modifications by achieving. an optimal surface geometry, which maximizes the effective surface area of the electrode while minimizing the after-potential polarization effect, thereby increasing charge transfer efficiency. This optimization is achieved by using a repeating geometric pattern, which can be represented in 2D by a sawtooth waveform with an amplitude equal to approximately ½ of the wavelength. If the 2D model of the surface with high geometric area is described as a sawtooth pattern with an electrical double layer formed equidistance from all surfaces, then at a sawtooth wavelength of less then the thickness of the double layer, no increase in capacitance would be seen. This would suggest that an optimum wavelength would be one which results in a surface which is optimally 45 degrees from the original surface, or alternatively, one which maximizes the amplitude of the waveform.

For signals having pulse widths within the range of interest, that is, approximately 0.5 ms to 5 ms in direction, the ideal surface geometry would consist of regular, trilateral pyramidal-shaped structures having an amplitude of between 250 and 400 nanometers. The angle between the sides of the pyramidal-shaped structures and the base of the structures would ideally be 45 degrees. As this perfect geometry may not be possible to produce in all instances, variations may produce electrical characteristics that are within acceptable ranges. For example, the angle between the sides of the pyramidal-shaped structures may vary from about 20 to about 70 degrees. Additionally, the base of the structures may be quadrilateral or polygonal in shape, but may also be composed of any combination of lines and curves, up to and including a completely circular base, resulting in a cone-shaped structure. The tops of the pyramidal-shaped structures would ideally be a sharp point, but the tops may also be truncated or curved, making the structures frustums.

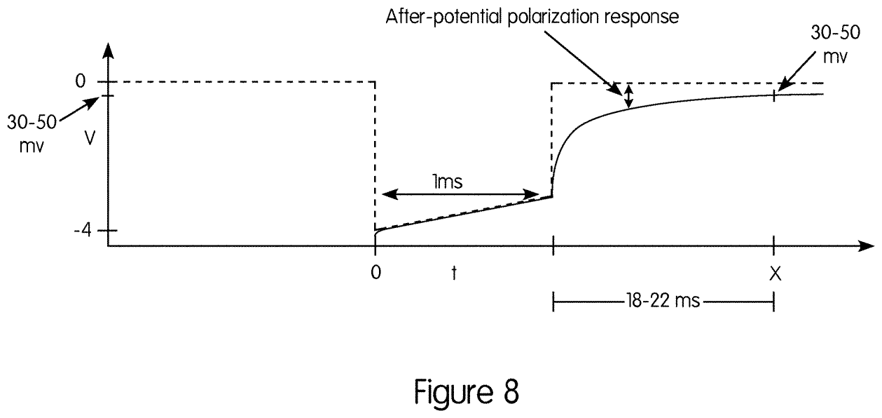

Electrically, it is desirable that the double layer capacitance be on the order of 70 mF/cm² or above. With respect to after-potential polarization, FIG. 8 shows a plot of after-potential polarization versus time for the preferred embodiment of the invention. It can be seen that, with a stimulation pulse of negative 4V, the voltage in the double layer capacitance drops to within 30 to 50 mV of its unstimulated level within 18-22 ms after the trailing edge of the stimulation pulse.

Because the repeating pattern of geometry is the predominant factor in enhancing electrical performance, it is optimum to produce this geometry on all surfaces which are to be used for stimulation and to closely pack this geometry, thereby reducing porous voids between the columnar structures. This results in a maximized performance electrode having the desired high surface area to promote high double layer capacitance and efficiency in signal transmission, while minimizing any after-potential polarization.

The method of this invention is currently best practiced using any one of a number of deposition processes, which can generally be described as physical vapor deposition processes, for the deposition of the coating. Various types of physical vapor deposition processes well know in the art include, but are not necessarily limited to, magnetron sputtering, cathodic arc, ion beam assisted PVD and LASER ablation PVD, any of which could be used to form the coating described herein. The method of the preferred embodiment is magnetron sputtering.

The invention may also be practiced by surface treatments which delete material from the surface, thereby forming the repeating geometric pattern with the necessary wavelength and amplitude. These methods include but are not limited to etching methods using chemicals, plasmas and lasers.

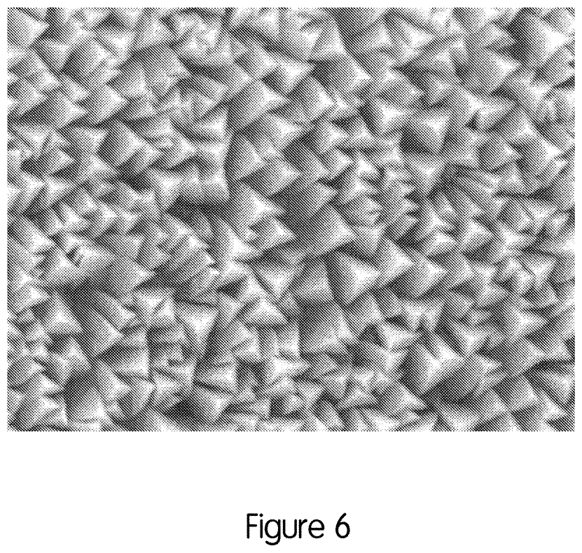

The preferred method for practicing the invention is a coating preferably formed using a primary metallic constituent and secondary reactive constituent which will combine with the metallic constituent to promote the growth of a [1, 1, 1] crystal structure. In the preferred embodiment, the primary metallic constituent is titanium, and the secondary reactive constituent is nitrogen, which forms a titanium nitride coating. In the preferred embodiment, approximately 90% plus of the surface of the coating was found to have the desired [1, 1, 1] crystal structure, evidenced by the formation of well-defined pyramidal-shaped protrusions on the surface of the coating, as shown in FIG. 6. It has been found, however, that acceptable electrical characteristics can be obtained with surfaces having as low as 80% [1, 1, 1] crystal structure on the surface of the coating.

The primary metallic constituent should be biocompatible, and the reactive constituent should form a compound with the primary that is electrically conductive, biostable, has anodic and cathodic corrosion resistance and has a cubic crystal structure which can grow in a [1, 1, 1] configuration. Examples of materials are nitrides, oxides and carbides of Ti, Ta, Nb, Hf, Zr, Au, Pt, Pd and W. In the preferred embodiment, titanium is the primary metallic constituent and nitrogen is the reactive constituent. This process will work with a substrate composed of any material, such as platinum, capable of reaching a temperature which permits diffusion and intermixing of the coating with the electrode surface.

During the coating process, the substrate is held at a temperature which allows surface diffusion prior to the coating condensate solidifying. This tends to result in larger or more diffuse nucleation sites, or may eliminate the nucleation sites in some instances. The surface diffusion promotes an intermixed layer where the electrode base material is in alloy or solid solution with the metallic constituent of the condensate.

In the preferred method the substrate temperature is held between approximately 20% and 40% of the melting point of the metallic coating species. In the preferred embodiment of this invention, the metallic coating species is titanium. This elevated temperature promotes diffusion of the materials.

For nicely-shaped pyramidal or tetragonal structures to be formed, it is desired that the plasma flux strike the surface at a very low angle, that is, the plasma flux should be coming in perpendicular to the surface of the device. On areas of the surface of a device where the plasma flux strikes the surface at an oblique angle, pyramidal or tetragonal structures having flattened tops are more likely to be formed, which will degrade the capacitive performance of the device.

To promote the growth of the coating of the present invention on devices of complex shape, it is therefore necessary to use a cylindrical target during the PVD process to ensure that all surfaces of the device receive plasma flux which is striking that surface on a perpendicular. Although all areas of the device will also have plasma flux striking at an oblique angle, the flux striking at an oblique angle tends to have less energy that that striking on a perpendicular, and therefore has more of an effect on the formation of the desired surface features.

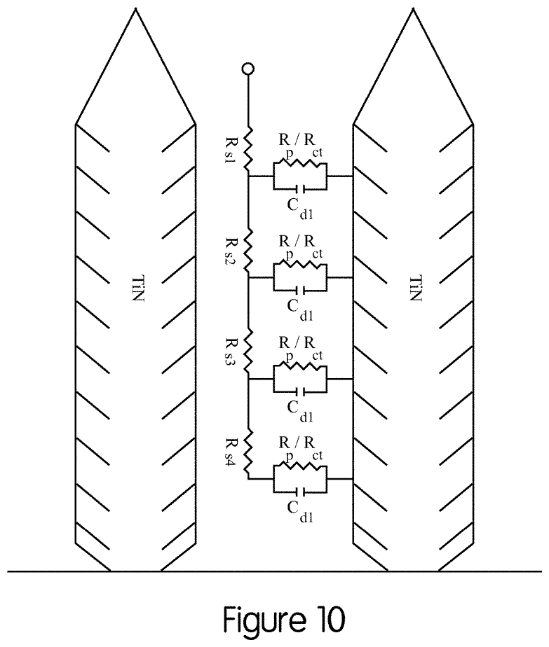

In one aspect of the invention, the surfaces of the electrodes are polished prior to the deposition of the coating using the PVD process. The polishing process reduces nucleation sites on the surface of the electrode where the columns of the structure of the coating would tend to grow, thus tending to make the columns closer together, thereby reducing porosities in the coating. This is shown in FIG. 10. This results in a structure wherein columns are tightly packed together, thereby reducing the porous voids between the columns where the resistance which contributes to the transmission line porosity effect is greatest. This resistance is modeled by resisters Rₛ₁, Rₛ₂, Rₛ₃ and Rₛ₄ in FIG. 10. Preferably, the surface would be polished to 11 micro-inches Ra or less, and preferably 8 micro-inches Ra or less.

In another aspect of the invention, the surface area of the coating should be maximized to maximize the double layer capacitance between the surface and the tissues of the body. Therefore, it is desirable that the sides of the pyramidal structures form a 45 degree angle with the plane of the base of the pyramid. However, for the preferred materials of which the coating is comprised, that being titanium nitride, the crystal structure will naturally form angles at approximately 65 degrees.

A 45 degree angle may be achieved by stressing the crystal during the formation process or by changing the materials of which the crystal. was made. However, subjecting the crystallites to stress to obtain the 45 degree angle may have a negative effect on the adhesion of the coating. Empirical analysis has determined however, that ranges as low as approximately 25 degrees to as high as approximately 65 degrees will work in an effective manner if the 45 degree angle is unable to be achieved. As a result, it is preferable not to attempt to modify the natural formation of a 65 degree angle when utilizing the preferred materials.

Another way to achieve increased surface area is to vary the amplitude of the geometry of the surface (i.e., the height of the peaks of the pyramidal shaped structures above a flat plane representing the base of the pyramids) on the surface of the coating. This can be achieved by varying the width of the columns, thereby changing the size of the base of the pyramids.

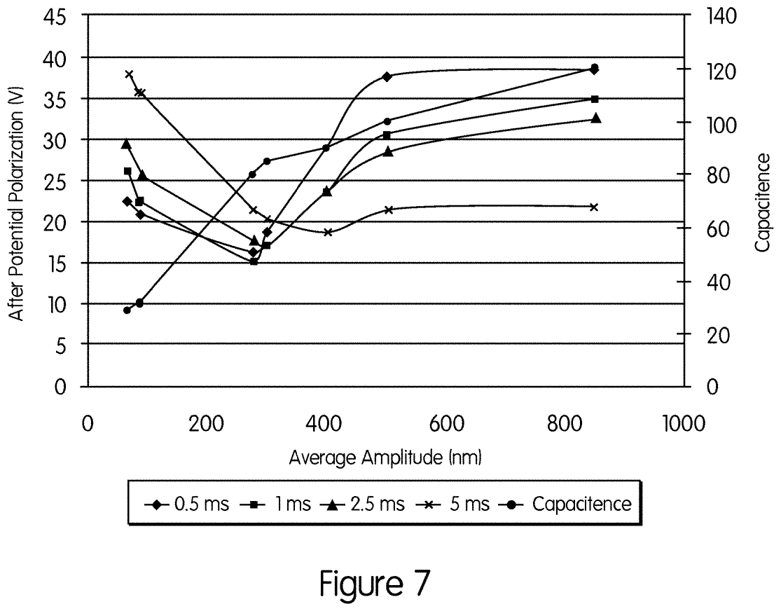

It has been found empirically that modifying the amplitude of the surface geometry to a certain height will result in a pyramidal structure having both acceptably high double layer capacitance and acceptably low after-potential polarization. FIG. 7 shows the amplitude of the surface geometry graphed against after-potential. polarization on the left axis and double layer capacitance on the right axis. The graph shows after-potential polarization values for signal wave durations ranging from 0.5 ms to 5 ms. The lowest points of after potential polarization at a given time after the trailing edge of the stimulation pulse occur between an average amplitude of 250 nm and 400 nm. It can also be seen that acceptable levels of double layer capacitance are obtainable with a surface having an average amplitude between 250 and 400 nanometers.

Although higher double layer capacitances are available at higher amplitudes of the surface geometry, the after-potential polarization also tends to rise to unacceptable levels at those amplitudes. The optimal range therefore appears to be between 250 and 400 nm.

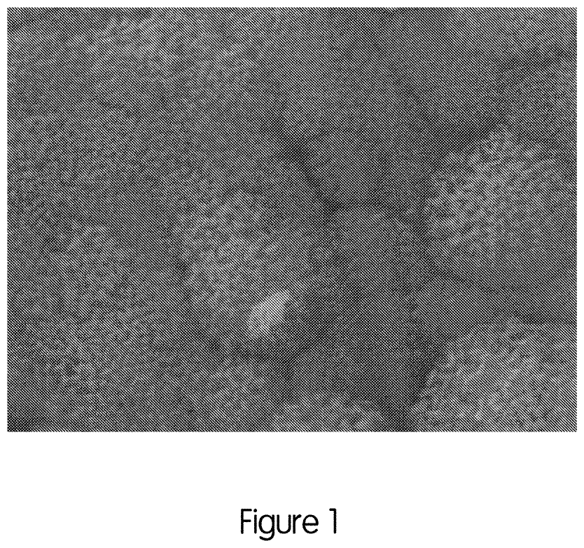

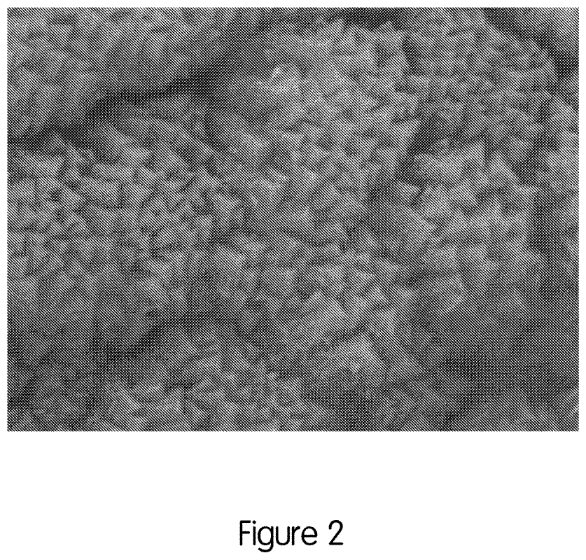

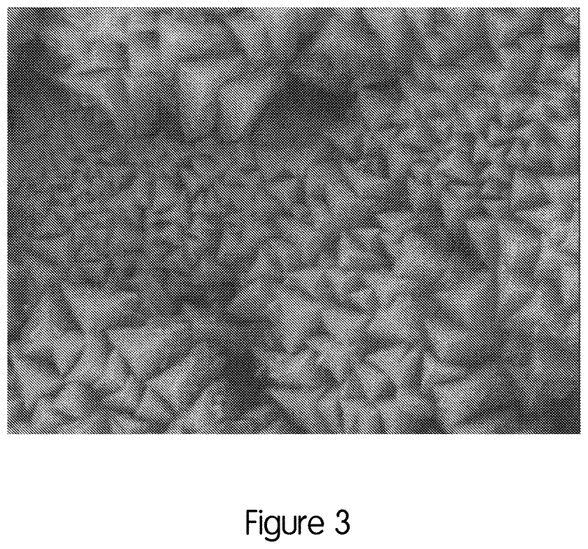

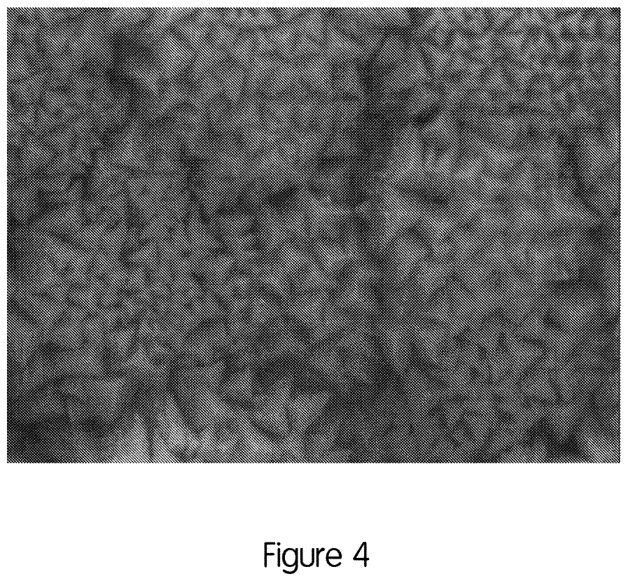

FIGS. 2 and 6 show surfaces having average amplitudes in the desired range (200-400 nm and 200-350 nm respectively). FIGS. 1, 3 and 4 show surfaces having the desired pyramidal. structure, but having an average amplitudes outside of the desired range of 250-400 nm, and therefore exhibiting unacceptable values for double layer capacitance, after-potential polarization, or both.

Because the angles in the formation of the crystallites are fixed, it is necessary to vary the width of the columns to vary the amplitudes of the crystallites. Changing the width of the columns has the effect of changing the size of the base of the pyramids, thereby resulting in a change in the height of the pyramids, if the angle between the sides and the base is kept constant.

In a physical vapor deposition process, the width of the columns can be varied by modifying the parameters under which the coating is deposited. The dominant factor is the pressure under which the deposition takes place. In general, the higher the pressure the narrower the column and the lower the pressure the wider the column. It is therefore necessary to choose a pressure, which may vary dependent upon the apparatus used to do the physical vapor deposition, which results in the column width which produces pyramids at the tops of the columns having average amplitudes in the desired range.

In addition, the power may also be varied, although the power, which affects the rate of deposition, is less of a factor and more difficult to control than the varying of the pressure. Changing the power will effect the rate of deposition. Generally, higher powers will produce wider columns.

The invention, which relates to the optimal surface geometry required to obtain the desired electrical characteristics, and various methods of obtaining that geometry is defined by the claims which follow.

I claim:

1. A method for optimizing a coating on a substrate comprising the steps of:
a. providing a primary metallic component
b. providing a secondary reactive component;
c. depositing said primary and said secondary components on said substrate such that deposited atoms of said secondary reactive component react with atoms of said primary metallic component prior to solidifying;
d. wherein the reaction of said primary metallic component and said secondary reactive component results in a surface having pyramidal or tetragonal crystal structures defined thereon; and
e. varying the deposition parameters such that the average amplitude of said crystal structures falls within a desired range.

2. The method of claim 1 wherein said varied deposition parameters are selected from a group consisting of pressure and power.

3. The method of claim 2 wherein said deposition takes place under a pressure that will result in average amplitude of said crystal structures being with said desired range.

4. The method of claim 3 wherein said primary metallic component is titanium, said secondary reactive component is nitrogen.

5. The method of claim 1 wherein said desired range for the average amplitude of said crystal structures is approximately between 250 and 400 nanometers.

6. The method of claim 1 wherein the sides of said pyramidal structures form an angle with the base of said pyramidal structures which is between 20 and 70 degrees.

7. The method of claim 6 wherein said angle is 45 degrees.

8. The method of claim 5 wherein the voltage on the double layer capacitance falls to within 30-50 mV of its unstimulated level with 18-22 ms of the trailing edge of the stimulation pulse.

9. The method of claim 5 wherein the double layer capacitance of said coating is approximately 70 mF/cm² or above.

10. The method of claim 1 further comprising the step of polishing said substrate prior to depositing said coating.

11. The method of claim 10 wherein said surface is polished to an Ra of 11 micro-niches or less.

12. The method of claim 10 wherein said surface is polished to an Ra of 8 micro-inches or less.

13. The method of claim 1 wherein said primary metallic component is selected from the group consisting of Ti, Ta, Nb, Hf, Zr, Au, Pt, Pd and W.

14. The method of claim 1 wherein said secondary reactive component is selected from a group consisting of nitrogen, oxygen and carbon.
